(19) United States
(12) Patent Application Publication
(10) Pub. No.: US 2006/0149190 A1
(43) Pub. Date: Jul. 6, 2006

(54) Attachment module for an injection device comprising an engagement control for a needle covering element

(76) Inventors: Philippe Kohlbrenner, Kaltacker (CH); Thomas Schuler, Sumiswald (CH); Martin Wittwer, Bowil (CH); Christoph Meier, Utzenstorf (CH); Peter Stettler, Kirchberg (CH)

Correspondence Address: DORSEY & WHITNEY LLP;INTELLECTUAL PROPERTY DEPARTMENT, 50 SOUTH SIXTH STREET, MINNEAPOLIS, MN, 55402-1498, US

(21) Appl. No.: 11/312,239

(22) Filed: Dec. 20, 2005

(30) Foreign Application Priority Data
Dec. 31, 2004 (DE) 10 2004 063 651.6

(51) Int. Cl. A61M 5/32

(52) U.S. Cl. 604/197

(57) ABSTRACT

An attachment module for use with an injection device, comprising an engagement control which comprises a blocking portion, a blocking member which engages with the engagement control and can be axially moved relative to the engagement control, wherein a movement of the blocking member relative to the engagement control is one of blocked or at least impeded at least in an axial direction when the blocking member is situated in the blocking portion.

### CROSS-REFERENCE TO RELATED APPLICATION(S)

This application claims the benefit of German Application No. DE 10 2004 063 651.6, filed on Dec. 31, 2004, the contents of which is incorporated herein by reference.

### BACKGROUND

The invention relates to devices for delivering, administering or dispensing measured or selected amounts of a substance, and to methods of their manufacture and use. More particularly, it relates to medical devices for administering a substance, including injection devices, syringes, pumps, etc. More particularly, it relates to an attachment module for use with an injection device, in particular an injection pen, which can expose a needle protected by a needle covering element, for making an injection.

Injection apparatus, in particular injection pens, allow a patient to independently self-administer a medicine, in most cases a fluid. For this purpose, it is necessary for the patient to pierce body tissue with a needle, inserting it into the skin. For a majority of patients, it generally requires a great effort to pierce themselves with a needle. This is particularly the case when the needle is visible, i.e., when injecting into the skin can be followed or observed. Some patients also dread the pain caused by injecting the needle into the skin.

It is generally known that, in order to inject a needle with less pain, the needle must penetrate quickly into the skin. In a self-injection using an injection pen, this is often only possible to a limited extent because on the one hand, there is no end stopper which can limit the penetration depth of the needle into the skin, and on the other hand, the injecting movement is performed by hand, which increases the patient's fear of causing pain.

A device into which an injection pen can be inserted and biased against the device by a spring is known from the prior art. In order to make an injection, the device is placed onto a desired point on the body and the pen, biased via the spring, is moved as a whole in the direction of the body such that a needle carried thereby is injected into the tissue.

U.S. Pat. No. 5,980,491 shows an automatic needle inserting device for a pen-shaped syringe comprising an ampoule holder and a dosing setting and injection part using which a desired dosage can be set and then expelled from the ampoule. The device includes a tubular casing in which a tubular syringe holder which can be connected to the syringe is arranged such that it can be axially shifted. The syringe holder can be shifted in a proximal direction in order to tense a spring, and then released for injecting, in order to be moved together with the pen-shaped syringe in a distal direction by the spring.

### SUMMARY

One object of the present invention is to provide a module for use with an injection device, in one embodiment an injection pen, wherein the module enables an injection needle to pierce the skin as painlessly as possible.

One embodiment of an attachment module in accordance with the present invention, for use with an injection device, comprises an engagement control which comprises at least one blocking portion and a blocking member which engages with the engagement control and can be axially moved relative to the engagement control. Axial movement or mobility should be understood to mean a movement generally along or in the direction of the longitudinal axis of the injection device.

In one embodiment of an the attachment module in accordance with the present invention, a movement of the blocking member relative to the engagement control is blocked or prevented or at least impeded at least in an axial direction when the blocking member is situated in the blocking portion. The attachment module can be fixedly connected to the injection device, such that the attachment module is a part of the injection device, but in some embodiments, the attachment module is preferably a separate part which can be fastened to the injection device.

The injection device and the attachment module can be supplied together, separate from each other in a set or already connected to each other. The attachment module which can be attached to the injection device can also be detached from the injection device. The injection device and/or the module therefore comprise means, such as a thread, a bayonet lock, etc., which enable the two components to be correspondingly and/or complementarily fastened to each other. The user of the injection device can thus decide for himself whether he wishes to perform an injection with or without the attachment module in accordance with the present invention. In principle, the attachment module could be connected to the injection device such that it is connected once and cannot be detached from the injection device without being destroyed.

In some embodiments, the attachment module preferably comprises a needle covering element which can at least partially surround a needle. The needle is advantageously carried by or a part of the injection device. The needle covering element is mounted, in particular by a casing, such that it can be shifted longitudinally, and in some preferred embodiments, cannot be rotated relative to the casing. The needle covering element may be, for example, coupled to one of the engagement control and the blocking member, wherein one is slaved in a movement of the needle covering element. In some embodiments, the needle covering element can be a needle protecting sleeve fixedly connected to one of the engagement control and the blocking member via a transfer means and, in some embodiments, forms the transfer member and the engagement control, or the blocking or locking member. The needle covering element and the engagement control or the blocking member can therefore be formed in one part. The other of the engagement control and the blocking member can be coupled to the casing, advantageously in one piece. In some preferred embodiments, the needle covering element is coupled to the engagement control and/or the casing is coupled to the blocking member.

In some embodiments, the needle covering element is moveable only far enough that a needle associated with the injection device cannot protrude beyond the distal end of the needle covering element when the blocking member is situated in a preparation portion of the engagement control. In principle, the needle covering element is dimensioned such that when the attachment module and the injection device are connected, the needle provided for the injection is covered by the needle covering element such that the patient preferably cannot unintentionally pierce himself with the needle. The needle may be completely surrounded by the needle covering element, wherein the needle covering element comprises an opening out of which the needle can emerge. The needle covering element and/or the attachment module may be adapted to a standard needle or needle length provided for the injection device. For applications in which a modified needle length is required, such as for example a shorter or longer length than the standard needle length, the needle covering element and/or the attachment module, in particular the engagement control, can be correspondingly adapted to the modified needle length.

In some embodiments, the engagement control with which the blocking member engages may be configured such that the blocking member can be moved in a preparation portion of the engagement control. The engagement control and, in particular, the preparation portion can be a contour along which the blocking member can slide. The path by which the blocking member can be moved in the preparation portion corresponds to the path by which the needle covering element can be moved without exposing the needle or a part of the needle. In a specific embodiment, the blocking member cannot be shifted in the preparation portion, since the length of the preparation portion is approximately equal to the length of the blocking member. The length of the blocking member is to be understood to mean the length by which the blocking member is in contact with the preparation portion. In some preferred embodiments, the preparation portion exhibits at least the length of the blocking member, preferably a greater length than the blocking member, such that the blocking member can slide along the preparation portion. The engagement control can advantageously comprise a blocking portion which borders the preparation portion. The position of the blocking portion in the engagement control is advantageously selected such that the needle or the tip of the needle does not protrude from the needle covering element when the blocking member is situated in the blocking portion. The position of the blocking portion can be adapted to a standardized needle or to a needle deviating from the standardised needle length. The needle covering element can be moved far enough that it does not expose the needle when the blocking member is situated in the preparation portion or in the blocking portion of the engagement control.

In a preferred embodiment, the preparation portion can be inclined in its length relative to the movement direction of the needle covering element and/or the longitudinal direction of the attachment module, in particular by a preparation portion angle. The longitudinal direction of the attachment module advantageously corresponds to the longitudinal direction of the injection device to which the attachment module can be attached. The movement direction of the needle covering element generally corresponds to the longitudinal direction of the attachment module. The preparation portion can be a curve or an inclined plane, the gradient or gradients of which are inclined relative to the longitudinal direction of the attachment module. The blocking member and the engagement control comprise a cam gear which converts a longitudinal movement of the engagement control relative to the blocking member into a movement in an additional direction, in particular a direction transverse to the movement direction of the needle covering element. In a relative sliding movement along the preparation portion, the blocking member is advantageously moved, additionally, transverse to the longitudinal direction.

The engagement control can also comprise an injection portion. The blocking portion advantageously limits the injection portion at one end. An injection stopper can be situated at the other end of the injection portion. When it is situated in the injection portion, the blocking member can slide back and forth between the blocking portion and the injection stopper, relative to the injection portion. The injection portion is approximately parallel to the movement direction of the needle covering element. The needle covering element may be moved far enough that a needle provided for the injection device protrudes beyond the distal end of the needle covering element when the blocking member is situated in the injection portion of the engagement control. The length of the injection portion or the path by which the blocking member can be shifted in the injection portion advantageously corresponds to at least the length by which the needle provided for the injection device can protrude beyond the end of the needle covering element. The injection stopper for the needle covering element can in particular be attached to the casing. The position of the injection stopper with respect to the longitudinal direction can determine the path by which the needle covering element can be pushed back and thus also the length by which a needle, in particular a standard needle provided for the injection device, can protrude from the needle covering element. When using needles which deviate from the length of the standard needles, i.e., which are longer or shorter than the standard needles, it is particularly advantageous to provide an attachment module in each case which is adapted in the length of the injection portion or the position of the injection stopper to the respective needles. The attachment modules for the respective needles, adapted to the different needle lengths, can advantageously be identified by an imprint, color code or similar means of identification known from the prior art.

In a preferred embodiment, the attachment module can comprise a needle covering element held by a spring element in an initial position covering a needle when no external force is exerted on the needle covering element. An external force is understood to mean a force which is directed counter to the spring force of the spring element and which serves to push the needle covering element toward the casing of the attachment module. In the initial position of the needle covering element, the blocking member is advantageously situated in a preparation portion of the engagement control. The engagement control can also exhibit a contour along which the blocking member can slide while the needle covering element is moved. The contour can be designed such that the blocking member at least temporarily engages with each of the preparation portion, the blocking portion and the injection portion when the needle covering element is moved from its initial position to a completely pushed-back position in which the needle covering element exposes the needle to its maximum injection length. When the casing of the attachment module forms the stopper responsible for the maximum injection length of the needle, the injection stopper can assume a position in which the blocking member does not engage with the injection stopper.

In some embodiments, the blocking member is preferably held by a spring element in or on the engagement control. This helps ensure that the blocking member engages with the engagement control. The blocking member can be moved or released from the blocking portion by an unblocking member, to enable the blocking member to engage with or be moved into the injection portion, whereby the needle can be exposed by the needle covering element. Advantageously, the blocking portion and the blocking member each form a co-operating stopper, wherein the two co-operating stoppers are transverse to the movement direction of the needle covering element or longitudinal direction of the attachment module. The stoppers can be at approximately right angles to the movement direction of the needle covering element. A blocking portion angle is advantageously formed between the normal on the movement direction and the stopper area of the blocking portion. In one embodiment, this can form an obtuse angle (i.e., an angle greater than 90°) between the area of the blocking portion and the area of the injection portion or, in some preferred embodiments, the area of the blocking portion and the area of the preparation portion.

In one preferred embodiment, the blocking portion is simultaneously an unblocking member. In particular, the two co-operating stoppers can slide off against each other in the blocking portion, due to an external force exerted on the needle covering element which is greater than the force of the spring element acting on the needle covering element. In particular, the blocking portion forms a gear area which co-operates with the blocking member and converts a longitudinal movement of the engagement control relative to the blocking member into an additional transverse movement of the blocking member, wherein relative to an equal axial path, the transverse path of the blocking member through the blocking portion is greater than that through the preparation portion. In some embodiments, the blocking portion angle is preferably large enough that the two co-operating areas of the blocking portion and the blocking member can slide off against each other when the needle covering element is moved. The two co-operating areas can thus not be self-locking. In particular, there is theoretically no self-locking if the blocking portion angle is greater than arctan

$$( \frac{1}{μ} ) ,$$

wherein μ indicates the coefficient of friction, for example the coefficient of sliding friction, preferably the coefficient of static friction of the two co-operating areas. The force necessary to release the blocking member from the blocking portion, which is in particular exerted on the needle covering element, is strongly dependent in this embodiment on the spring force with which the blocking member is held in the engagement control or blocking portion.

In accordance with this embodiment, the needle is injected using the injection device connected to the attachment module in accordance with the invention, such that the distal end of the needle covering element is placed onto the desired point on the body and the user of the device, who has grasped the device, exerts an axial force on the device in the injection direction. This moves the blocking member relatively along the preparation portion into the blocking portion of the engagement control. The movement of the needle covering element into the casing of the attachment module or the movement of the injection device in the injection direction is blocked or impeded when the blocking member is situated in the blocking portion. When the force in the injection direction is increased, the blocking member is pressed out of the blocking portion by the blocking portion co-operating with the blocking member, which moves the blocking member into the injection portion, such that the needle covering element is abruptly released and abruptly pushed back into the casing by the increased, sustained force from the user of the device. The needle is thus pulse-injected or “shot” into the body tissue, and the user merely feels a small prick. While the needle covering element is slid completely into the casing, the blocking member slides relatively along the injection portion until the injection stopper blocks the needle covering element from moving further. The needle is then injected into the skin of the patient to the desired length.

In another preferred embodiment, the two co-operating stoppers are self-locking against sliding off in the blocking portion, due to an external force exerted on the needle covering element. Theoretically, there is self-locking when the blocking portion angle is smaller than arctan

$$( \frac{1}{μ} ) ,$$

wherein μ indicates the coefficient of friction, for example the coefficient of sliding friction, in particular the coefficient of static friction of the two co-operating areas. This means that it is theoretically not possible for the blocking member to slide off out of the blocking portion, irrespective of the force exerted on the needle covering element. In practical terms, the blocking member could potentially be released in this embodiment, if for example a force in the distal direction, exerted on the needle covering element, is greater than it would be when normally or appropriately arranged, since elastic deformations of the components would become significant as the force increases. The invention, however, is to be viewed from the perspective of it being used appropriately.

An unblocking member can be provided for releasing the blocking member from the blocking portion. The unblocking member can be connected to the blocking member. In some embodiments, the unblocking member can comprise a gear area which can co-operate with a gear area of a releasing member such that in a releasing movement of the releasing member, the blocking member is released from its engagement with the engagement control, i.e., from its blocking engagement with the blocking portion. In some embodiments, the releasing member can preferably be moved translationally. The translational direction of the releasing member corresponds approximately to the longitudinal direction of the attachment module or of the injection device. In one embodiment, the releasing member can be a slider which is mounted by the casing of the attachment module such that it can be moved longitudinally, or it can comprise a gear area at its distal end, wherein the gear area advantageously runs or extends obliquely with respect to the movement direction of the releasing member. The gear area of the releasing member can run in the sense of an inclined plane.

The releasing member in accordance with this embodiment comprises a gear area which is advantageously inclined relative to the longitudinal direction of the attachment module or the translational direction of the releasing member. The angle between the gear area of the releasing member and the longitudinal direction of the attachment module or translational direction of the releasing member may be referred to as the releasing member angle. The angle between the angular area of the unblocking member and the longitudinal direction of the attachment module or translational direction of the releasing member may be referred to as the unblocking member angle. In some embodiments, the unblocking member angle and the releasing member angle are preferably approximately of the same size in a position in which the releasing member can engage with the unblocking member, whereby the gear areas can substantially slide off on each other. The releasing movement of the releasing member can be a translational movement of the releasing member in the distal direction. The releasing movement of the releasing member moves the unblocking member and advantageously also the blocking member, via the co-operating gear areas, transversely to the translational direction of the releasing member, in particular with a pivoting movement. The transverse or pivoting movement of the blocking member can move the blocking member out of its engagement with the engagement control. The gear area of the releasing member and the gear area of the unblocking member can advantageously co-operate only when the blocking member is situated in the blocking portion. The blocking member can not be moved out of its engagement with the engagement control when the blocking member is situated in the blocking portion. This helps ensure that the needle covering element or attachment module together with the injection device has to be pressed onto the point on the body before the blocking member can be released from the engagement control.

The releasing member can be a part of the attachment module. In this case, the releasing movement of the releasing member can be triggered by a push button or sliding button connected to the releasing member and protruding out of the casing of the attachment module. The releasing member can also be part of the injection device, wherein the releasing member can be inserted into the attachment module. In some embodiments, the releasing member preferably consists of two parts, wherein one part is formed on or in the attachment module and one part is formed on or in the injection device. The two parts of the releasing member can be designed such that they cannot fall out of the attachment module or the injection device when the attachment module is detached from the injection device. When the attachment module is combined with the injection device, the at least two parts can co-operate such that they can jointly perform a releasing movement. It is therefore advantageous to provide a transfer or connecting member on each of the two parts of the releasing member, which co-operates with the other of the two releasing members. For a purely sliding transfer, a transfer or connecting means could in principle be two co-operating spur areas. A triggering element can advantageously be provided on or in the injection device, using which the releasing movement can be performed or triggered.

In another preferred embodiment, the releasing member can be connectable or connected to the drive module or the drive and dosing module of the injection device, such that the releasing member is slaved by a drive movement or triggering movement, typically a movement in the longitudinal direction of the drive module or the drive and dosing module. It can be advantageous if the releasing member engages with a corresponding part of the drive module or the drive and dosing module, such that the releasing member is slaved in a translational movement of the corresponding part. The drive module or the drive and dosing module causes the releasing movement of the releasing member before a drive movement acting on the piston in the ampoule of the injection device is triggered. This advantageously results in the needle being injected into the patient first, before a product is delivered or administered, or in the needle already being injected into the patient's body when the product is delivered.

When a drive module or drive and dosing module of the injection device is activated, the releasing member engages with the unblocking member, which releases the engagement of the blocking member from the blocking portion, the needle covering element exposing the needle.

In some embodiments, the blocking element can be formed on an arm which is elastic transverse to the longitudinal direction. The arm can be formed in one part with the casing. The transition point at which the arm is connected to the casing is designed such that the transition point forms a spring element. In principle, however, the transition point could also be formed to be approximately rigid and the arm formed to be elastic, such that the arm forms the spring element. It has proven advantageous to arrange two blocking elements such that they respectively engage in approximately opposite directions with one engagement control each. One unblocking member can be respectively assigned to each blocking member. One each of a blocking member and unblocking member can advantageously be arranged on an arm which is elastically connected to the casing. In one preferred embodiment, the two oppositely engaging blocking members can be arranged on one arm each, wherein the two arms can meet in a connecting point with the casing and spring oppositely along a movement line. This can advantageously provide two gear areas on a releasing member, each of which can co-operate with a gear area of the unblocking members. Thus, for example, the gear areas of the releasing member can be arranged in a V shape in the longitudinal direction. The gear areas of the unblocking members can also be arranged in a V shape in the longitudinal direction, wherein the gear areas of the releasing member arranged in a V shape can advantageously be slid over the gear areas of the unblocking members arranged in a V shape, which can release all the blocking members. The elastic arms can also be arranged in a V shape, wherein the elastic transition point to the casing can be formed at the tip of the V. In yet another embodiment, a number of the engagement controls in accordance with the invention, each with a co-operating blocking member, can be distributed over the circumference of the casing of the attachment module. Two V-shaped arrangements of the arms forming the blocking members and, as applicable, the unblocking members, diametrically opposing on the casing of the attachment module, have proven suitable. It is correspondingly advantageous to assign a releasing member, with gear areas arranged in a V shape, to each V-shaped arrangement. The described mechanism of the attachment module can be covered by an outer cover to protect the mechanism from being inappropriately accessed or damaged.

In one exemplary embodiment, an injection device provided with the attachment module can be used with a releasing member or a self-locking blocking portion. The user of the device grasps the device and presses the distal end of the needle covering element onto a point on the body provided for an injection. By exerting an axial movement on a triggering element, e.g., a dosing button, the user can in principle translationally move the releasing member and then trigger a drive mechanism for administering the product in the ampoule. As long as the blocking member is situated in the preparation portion, however, the releasing movement of the releasing member cannot take place, since the gear area of the releasing member cannot pass into engagement with the gear area of the unblocking member. By pressing the device onto the surface of the body, the preparation portion can slide along relative to the blocking member until the blocking member has reached the blocking portion of the engagement control. Due to the self-locking, the blocking member cannot slide out of the blocking portion due to an increase in the force on the needle covering element. If the blocking member is situated in the blocking portion, then the gear areas of the releasing member and the blocking member can co-operate such that the releasing movement of the releasing member moves the blocking member out of the blocking portion. The needle covering element is then abruptly pushed back into the casing of the attachment module due to the applied external force on the combined device, and the needle is injected into the body. As the needle is injected, the injection portion slides relatively along the blocking member until the injection stopper prevents the needle covering element from moving further into the casing. If the needle is drawn out of the body with the injection device, the needle covering element is pushed back into its initial position by the spring element. The blocking member or blocking members again engage with the preparation portion or with the respective preparation portions, wherein due to the resetting movement of the blocking member transverse to the longitudinal direction of the attachment module, the unblocking member presses the releasing member out of its engagement with the unblocking member.

The invention also relates to an injection device, comprising: a casing, a drive module or drive and dosing module accommodated by the casing, an ampoule which can be inserted and comprises a piston which can be shifted in the ampoule and on which the drive module or drive and dosing module acts, and a needle which can be fastened to the ampoule, wherein the injection device comprises an attachment module or can be connected to an attachment module comprising a needle covering element and an engagement control

### BRIEF DESCRIPTION OF THE DRAWINGS

FIG. 1 is a three-dimensional view of an attachment module in accordance with the present invention, in a longitudinal section;

FIG. 2 is a two-dimensional sectional representation of an injection device in accordance with the present invention, comprising an attachment module in accordance with the invention;

FIG. 3 depicts engagement controls in accordance with the present invention, each comprising a blocking member;

FIG. 4 depicts engagement controls in accordance with the present invention, each comprising a blocking member and an unblocking member, as well as a releasing member; and

FIG. 5 depicts an engagement control in accordance with the present invention, comprising a preparation portion running or extending approximately parallel to the longitudinal axis.

### DETAILED DESCRIPTION OF THE DRAWINGS

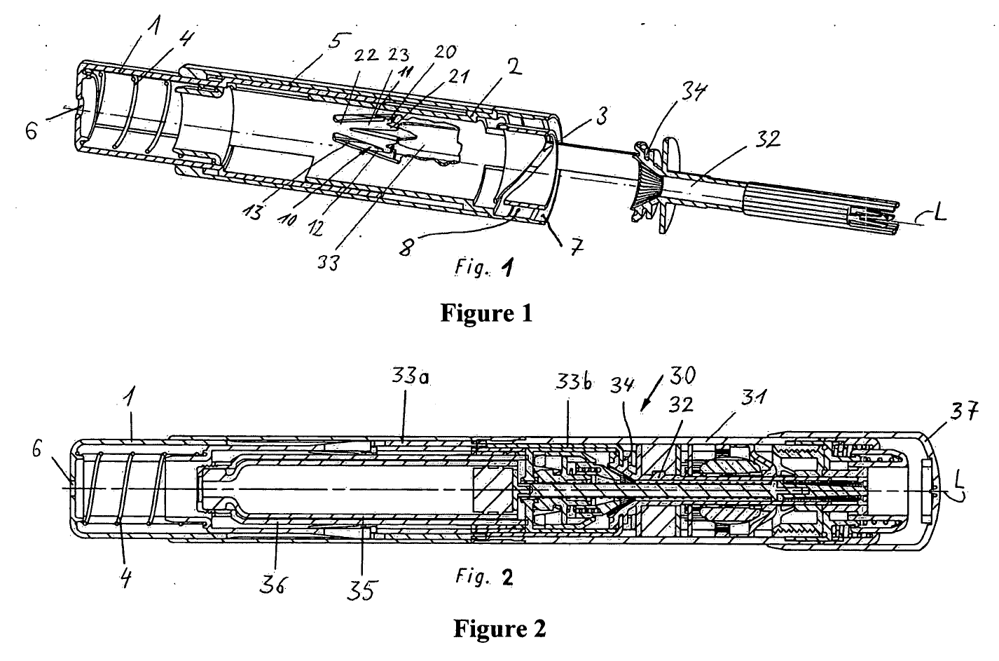

FIG. 1 shows a three-dimensional representation of an attachment module in accordance with the invention, in longitudinal cross-section. The attachment module comprises a casing 2 and a needle covering element 1 guided by the casing 2. The needle covering element 1 is secured against rotating and can be moved longitudinally relative to the casing 2. FIG. 1 shows the needle covering element 1 in an initial position in which no external force is acting on the needle covering element 1. The needle covering element 1 is therefore pressed in a distal direction, i.e., its initial position, by the spring element 4, for example a pressure spring. In the example shown, the needle covering element 1 is a needle protecting sleeve 1 which comprises an outlet opening 6 at its distal end for a needle (not shown). The pressure spring 4 is supported on the one hand on the inner side of the needle protecting sleeve 1 and on the other hand on an annular heel of the casing 2. The proximal end of the casing 2 comprises a locking element 3 in the form of a groove of a bayonet lock 3. The proximal end of the casing 2 also comprises a connecting diameter 7 which can be slid over a connecting diameter of the injection device, and a connecting catch 8 which, when the attachment module is completely slid onto the injection device 30, engages with an element on the injection device which can co-operate with the connecting catch 8, such that the attachment module can be prevented from being unintentionally detached from the injection device 30. In the embodiment shown, the attachment module is detachably fastened to the injection device 30.

A window is formed on the casing 2, in which two arms 23 are arranged in a V shape and fastened to the casing 2. The two arms 23 are connected to the casing 2 by the transition portion 22. In one embodiment, the transition portion 22 is designed such that the arms 23 can spring towards and away from each other. A blocking (or locking) member 20 is formed on each of the two arms 23 on their sides facing away from each other. The blocking member 20 in the form of a cam. The proximal ends of each of the arms 23 also comprise an unblocking (or unlocking) member 21. For the sake of simplicity, one arm together with its blocking member and its unblocking member is described in the following, since the same applies to the other arm. In the attachment module shown in FIG. 1, the part of the attachment module cut away in this view also comprises such an arrangement of arms, and that which is said regarding the two arms shown applies similarly to said arrangement of arms.

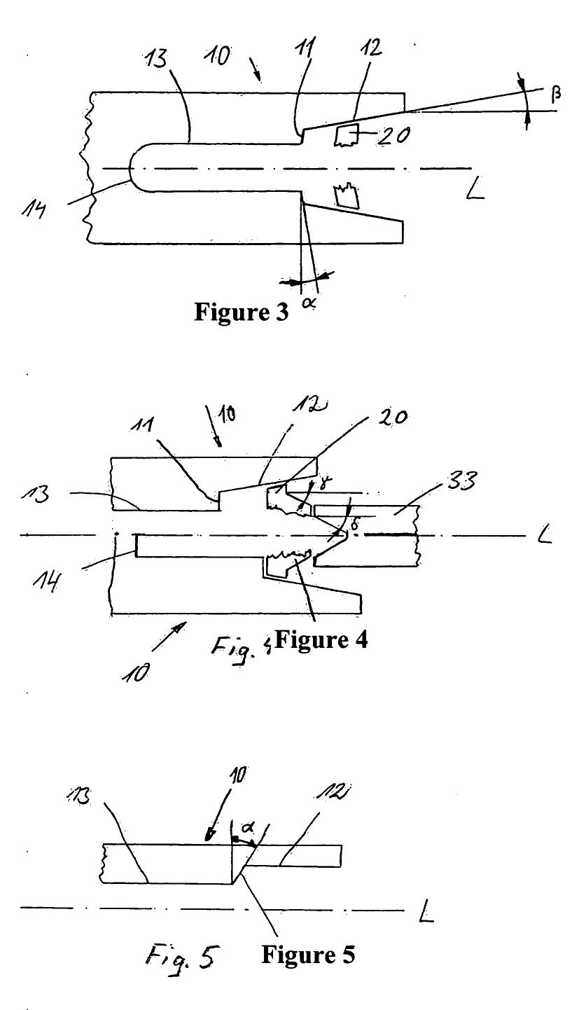

The arm 23 can be pivoted with a blocking member 20 and the unblocking member 21 about the connecting point 22. An engagement control 10 is connected to the needle covering element 1. The needle covering element 1 forms the engagement control 10. For each arm 23 or blocking member 20, an engagement control 10 is respectively provided, with which the blocking member 20 can engage. In particular, the blocking member 20 is pressed via the arm 23 into the engagement control 10 by the spring force of the transition portion 22. The engagement control 10 comprises a preparation portion 12, a blocking (or locking) portion 11 and an injection portion 13, as can also be seen from FIGS. 3, 4 and 5.

When the needle covering element 1 is moved in the proximal direction, the engagement control 10 is moved along the blocking member 20.

With reference to FIGS. 3 and 1, in the embodiment shown, the preparation portion 12 of the engagement control 10 is inclined relative to the longitudinal direction L by the angle β. The engagement control 10 can be moved along the longitudinal direction L. When the engagement control 10 is moved along the longitudinal direction L, the blocking member 20—which is substantially fixed in the longitudinal direction L—is moved inwards by the preparation portion 12. The engagement control 10 and the needle covering element 1 coupled to it can be moved in the proximal direction until the blocking portion 11 abuts the blocking member 20.

The blocking portion 11 forms a blocking portion angle α between its stopper area and the vertical onto the longitudinal direction L. The blocking portion angle α can for example assume the value of 0°, as shown in FIG. 4. Lastly, FIG. 3 also shows an injection portion 13 which extends approximately parallel to the longitudinal direction L and abuts the blocking portion 11. As shown in FIGS. 3 and 4, the injection portion 13 can be abutted by an injection stopper 14 which can limit the path by which the blocking member 20 is pushed in the injection portion 13 and thus also the path by which the needle covering element 1 can be distally pushed back into the casing 2.

As shown in FIG. 3, the blocking portion 11, formed as a stopper for the blocking member 20, can run or extend obliquely with respect to the longitudinal direction and thus form a blocking portion angle α. As shown by way of example in FIG. 4, the angle α can be very small or zero, such that when a force is exerted in the longitudinal direction L, the stopper area of the blocking portion 11 and the stopper area of the blocking member 20 co-operating with it prevent the respective stopper areas from sliding off on each other, irrespective of the size of the force applied. This case is referred to as self-locking. If, as shown by way of example in FIG. 5, the blocking portion angle α assumes a large value, then the co-operating areas of the blocking portion 11 and the blocking member 20 can slide off on each other due to a force, acting in the proximal direction, applied to the needle covering element 1 and thus to the engagement control 10. The blocking portion angle α would then have a value which does not cause the co-operating areas of the blocking portion 11 and the blocking member 20 to be self-locking. In accordance 10 with the blocking portion angle α shown by way of example in FIG. 5, which is not self-locking, the blocking portion 11 simultaneously forms an unblocking member 21 with which the blocking member 20 can be moved out of engagement with the blocking portion 11.

In some embodiments, the preparation portion 12 generally exhibits a smaller inclination relative to the longitudinal direction L than the blocking portion 11. Correspondingly, the force necessary to move the needle covering element 1 is less when the blocking member 20 is situated in the preparation portion 12 than when the blocking member 20 is situated in the blocking portion 11. The preparation portion 12 can extend approximately parallel to the longitudinal direction L, as shown in FIG. 5, wherein this is particularly useful for a blocking portion angle α which is not self-locking. In the following, the invention is explained by referring to FIGS. 1, 3 and 5, wherein a blocking portion angle α is provided which is not self-locking, whereby the blocking portion 11 simultaneously forms the unblocking member 21. The user of the injection device 30 with the attachment module attached (which may also be referred to as the combined device) grasps the combined device and presses the proximal end of the needle covering element 1 onto the point on the body provided for an injection. This moves the needle covering element 1 back slightly, the blocking member 20 and the preparation portion 12 of the engagement control 10 performing a movement relative to each other until the blocking member 20 abuts with the blocking portion 11. The movement of the needle covering element 1 is thus stopped. If the user of the device then increases the force with which the combined device is pressed onto the point 30 on the body, then the blocking member 20 is released from its engagement with the blocking portion 11, i.e., moved into the injection portion 13, by the blocking portion 11 which is not self-locking. If the blocking member 20 has been pressed completely out of the blocking portion 11 and moved into the injection portion 13, then the movement of the needle covering element 1 in the proximal direction is abruptly released, whereby the combined device except for the needle covering element 1 “shoots” in the distal direction and the needle is injected into the skin. The depth to which the needle is injected into the skin can be adapted by the length of the injection portion 13 extending in the longitudinal direction, i.e., by the distance from the blocking portion 11 which the injection stopper 14 exhibits in the longitudinal direction L. The injection stopper 14 can assume a semicircular shape, as shown in FIG. 3. The blocking portion assumes the form of an area running transverse to the longitudinal direction L, as shown by way of example in FIG. 4. In principle, the injection stopper 14 need not necessarily be attached to the injection portion 13 or engagement control 10. Similarly, the injection stopper 14 can be formed by a corresponding stopper area between the needle covering element 1 and the casing 2.

Referring to FIGS. 1, 2 and 3, the present invention may also be described on the basis of another exemplary embodiment. The user of the device presses the distal end of the needle covering element 1 of the combined device onto the point on the body provided for the injection. This slides the blocking member 20 relatively along the preparation portion 12 until the blocking member 20 passes into a blocking engagement with the blocking portion 11 of the engagement control 10. For this described embodiment, a preferred blocking portion angle α is one which causes self-locking between the co-operating areas of the blocking portion 11 and the blocking member 20. If the blocking portion 11 and the blocking member 20 are abutting, the blocking member 20 cannot be released from the blocking portion 11 by an increased pressing force by the combined device on the body. In order to release the blocking engagement (or lock), an unblocking member 21 is provided which is proximally situated on the blocking member 20 or the arm 23. The unblocking member 21 comprises a gear area which is inclined with respect to the longitudinal direction L by an unblocking member angle γ. A releasing member 33 is additionally provided which comprises a gear area at its distal end, said gear area being inclined relative to the longitudinal direction L by the releasing member angle δ. The releasing member angle δ and the unblocking member angle γ preferably correspond to the same order of magnitude. As shown in particular in FIG. 4, the respective gear area of the unblocking member 21 and of the releasing member 33 cannot be moved into engagement by moving the releasing member 33 in the distal direction when the blocking member 20 is situated in the preparation portion 12, and/or can be moved into engagement when the blocking member 20 is situated in the blocking portion 11. If the blocking member 20 is situated in the preparation portion 12, then the releasing member 33 cannot be moved in the distal direction since a spur area of the releasing member 33 abuts a spur area of the unblocking member 21 and a longitudinal movement of the releasing member 33 can thus be prevented.

If, however, the blocking member 20 is situated in the blocking portion 11, then the blocking member 20 and the unblocking member 21 have been moved inwards by the inclination of the preparation portion 12, as shown in particular in the lower half of FIG. 4, such that the gear areas of the releasing member and the unblocking member are moved into engagement in the distal direction by means of a longitudinal movement of the releasing member 33, wherein the movement of the releasing member 33 in the distal direction generates a transverse movement of the blocking member 20 inwards and moves the blocking member out of engagement with the blocking portion 11. The blocking member 20 is then situated in the injection portion 13, such that the needle covering element 1 can be pushed back and the blocking element 20 slide along the injection portion 13 until the blocking member 20 abuts the injection stopper 14. As already described, this releases the needle and injects it into the patient's skin.

By drawing back the combined device, the needle is drawn out of the patient's body and the needle covering element 1 is pressed in the distal direction by the spring 4. The movement of the needle covering element 1 in the distal direction moves the engagement control 10 along the blocking member 20 in the distal direction. When the blocking member 20 is situated in the region of the preparation portion 12, it springs off in the direction of the preparation portion 12 due to the elastic arrangement of the transition portion 22 and the arm 23, whereby the releasing member 33 is moved back in the proximal direction by the gear areas of the unblocking member 21. The releasing process for the needle covering element can then begin anew.

The releasing member 33 (which might be thought of and/or referred to as a key) can be a part of the attachment module and/or a part of the injection device 30. Advantageously, the releasing member 33 is accommodated in the casing 2 such that it can be longitudinally shifted, as shown in FIG. 1. The drive member 32 shown in FIG. 1 is not part of the attachment module, but rather of the injection device 30. In accordance with FIGS. 1 and 2, the releasing member 33 or the releasing member 33a, 33b formed in two parts engages with an annular groove 34 of the drive member 32 in the injection device 30. Activating, e.g., pressing on, a triggering element 37 formed as a triggering button moves the drive member 32 in the distal direction, which can move the releasing member 33 engaging with the annular groove 34 or the part 33b of the releasing member 33a, 33b engaging with the annular groove 34 in the distal direction if the gear areas of the unblocking member 21 and the releasing member 33 can be moved into engagement. The drive member 32 is part of a drive module or drive and dosing module which is accommodated in a casing 31 of the injection device. An ampoule holder 36 connected to the casing 31 of the injection device is situated distally on the casing 31 and can accommodate an ampoule 35 in its interior which can for example contain a medicine. The drive module or drive and dosing module acts on the one hand on releasing member 33 and on the other hand on a piston of the ampoule 35. Preferably, activating triggering element 37 first, if possible, releases the needle covering element via the releasing member, such that the needle can be injected. Only if this step of the releasing member has taken place can the piston then be moved in the delivery direction, in particular by an automatic drive.

Embodiments of the present invention, including preferred embodiments, have been presented for the purpose of illustration and description. They are not intended to be exhaustive or to limit the invention to the precise forms and steps disclosed. Obvious modifications or variations are possible in light of the above teachings. The embodiments were chosen and described to provide the best illustration of the invention and its practical application, and to enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

1. An attachment module of or for an injection device, comprising:
a) an engagement control which comprises a blocking portion;
b) a blocking member which engages with the engagement control and can be axially moved relative to the engagement control; wherein
c) a movement of the blocking member relative to the engagement control is blocked or at least impeded at least in an axial direction when the blocking member is situated in the blocking portion.

2. The attachment module according to claim 1, wherein the attachment module comprises a needle covering element which can at least partially surround a needle and is coupled to one of the engagement control and the blocking member, wherein one of the engagement control and the blocking member is slaved in a movement of the needle covering element.

3. The attachment module according to claim 2, wherein the needle covering element is a needle protecting sleeve.

4. The attachment module according to claim 2, wherein the needle covering element is coupled to the engagement control.

5. The attachment module according to claim 2, wherein the other of the engagement control and the blocking member is coupled to a casing.

6. The attachment module according to claim 2, wherein the needle covering element can be moved only far enough that a needle provided for the injection device cannot protrude beyond the distal end of the needle covering element when the blocking member is situated in a preparation portion of the engagement control.

7. The attachment module according to claim 6, wherein the preparation portion exhibits at least the length of the blocking member such that the blocking member can slide along the preparation portion.

8. The attachment module according to claim 7, wherein the preparation portion is limited at one end by the blocking portion.

9. The attachment module according to claim 6, wherein the preparation portion is inclined in its length relative to the longitudinal direction of the attachment module.

10. The attachment module according to claim 7, wherein in a sliding movement along the preparation portion, the blocking member is moved transverse to the longitudinal direction.

11. The attachment module according to claim 1, wherein the engagement control comprises an injection portion limited at one end by the blocking portion.

12. The attachment module according to claim 11, wherein the needle covering element can be moved far enough that a needle associated with the injection device protrudes beyond an end of the needle covering element when the blocking member is situated in the injection portion of the engagement control.

13. The attachment module according to claim 12, wherein one of the length of the injection portion or the path over which the blocking member can be shifted in the injection portion corresponds to at least the length by which the needle can protrude beyond the end of the needle covering element.

14. The attachment module according to claim 10, wherein the injection portion is approximately parallel in its length to the longitudinal direction.

15. The attachment module according to claim 1, further comprising a needle covering element held by a spring element in an initial position covering a needle when no external force is exerted on the needle covering element.

16. The attachment module according to claim 15, wherein in the initial position of the needle covering element, the blocking member is situated in a preparation portion of the engagement control.

17. The attachment module according to claim 1, wherein the engagement control forms a contour along which the blocking member can slide while a needle covering element is moved.

18. The attachment module according to claim 17, wherein the blocking member is held by a spring element in or on the engagement control.

19. The attachment module according to claim 1, wherein the blocking portion and the blocking member each form a co-operating stopper, wherein the co-operating stoppers are transverse to the longitudinal direction of the attachment module.

20. The attachment module according to claim 1, wherein the blocking portion is simultaneously an unblocking member.

21. The attachment module according to claim 19, wherein the co-operating stoppers can slide against each other in the blocking portion, due to an external force exerted on the needle covering element which is greater than the force of the spring element.

22. The attachment module according to claim 19, wherein the co-operating stoppers are self-locking against sliding off in the blocking portion, due to an external force exerted on the needle covering element.

23. The attachment module according to claim 1, wherein an unblocking member is connected to the blocking member.

24. The attachment module according to claim 23, wherein the unblocking member comprises a gear area which can co-operate with a gear area of a releasing member such that in a releasing movement of the releasing member, the blocking member is released from its engagement with the engagement control.

25. The attachment module according to claim 24, wherein the gear area of the releasing member and the gear area of the unblocking member co-operate only when the blocking member is situated in the blocking portion.

26. The attachment module according to claim 24, wherein the releasing member is guided in the longitudinal direction.

27. The attachment module according to claim 24, wherein the releasing member is a part of the attachment module.

28. The attachment module according to claim 24, wherein the releasing member is a part of the injection device.

29. The attachment module according to claim 24, wherein the releasing member comprises at least two parts, wherein one part is formed on or in the attachment module and one part is formed on or in the injection device and when the attachment module is combined with the injection device, the at least two parts co-operate such that they can jointly perform a releasing movement.

30. The attachment module according to claim 24, wherein the injection device comprises a drive module and wherein the releasing member can be or is connected to the drive module, such that the releasing member is slaved by a drive movement.

31. The attachment module according to claim 23, wherein when a drive module or drive and dosing module of the injection device is activated, the releasing member engages with the unblocking member, which releases the engagement of the blocking member from the blocking portion, the needle covering element exposing the needle.

32. An injection device, comprising:
a) a casing;
b) a drive module and dosing module accommodated by the casing;
c) an ampoule which can be inserted in the casing and comprises a piston which can be shifted in the ampoule and on which the drive and dosing module acts;
d) a needle which can be fastened to the ampoule; and
e) an attachment module comprising an engagement control which comprises a blocking portion, a blocking member which engages with the engagement control and can be axially moved relative to the engagement control, wherein a movement of the blocking member relative to the engagement control is one of blocked or at least impeded at least in an axial direction when the blocking member is situated in the blocking portion.
